(19) United States
(12) Patent Application Publication
(10) Pub. No.: US 2025/0064680 A1
(43) Pub. Date: Feb. 27, 2025

(54) AN OSTOMY COLLECTION VOLUME STAND

(71) Applicant: SALTS HEALTHCARE LIMITED, West Midlands (GB)

(72) Inventors: Lee Howard, West Midlands (GB); Ewa Jasicka, West Midlands (GB); Lee Smith, West Midlands (GB)

(21) Appl. No.: 18/728,773

(22) PCT Filed: Jan. 19, 2023
(86) PCT No.: PCT/GB2023/050087

§ 371 Date: Jul. 12, 2024

(30) Foreign Application Priority Data
Jan. 19, 2022 (GB) 2200649.8

(51) Int. Cl. A61J 1/16; A61F 5/445; A61J 1/10

(52) U.S. Cl. CPC A61J 1/16; A61F 5/445; A61J 1/10

(57) ABSTRACT

An ostomy collection volume stand for supporting a urostomy collection volume including: a support arrangement having a first condition and a second condition, wherein in the first condition the support arrangement provides an enclosed storage volume for storing one or more urostomy collection volumes and in the second condition the support arrangement provides a support formation is configured to engage with a portion of a urostomy collection volume such that the support arrangement provides a support for the urostomy collection volume when it is in use.

### INTRODUCTION

Embodiments of the invention relate to an ostomy collection volume stand for supporting a urostomy collection volume.

Ostomy appliances are well known medical devices for users that have a stoma. Generally, the appliance has an opening for the stoma to be received in and an adhesive member that contacts the body and holds the appliance in position. Waste exiting the stoma is collected in a collection volume which is formed from two flexible walls connected together. Often the appliance includes an outlet valve, so that a user can empty waste that is collected in the collecting volume without having to remove and dispose of the entire appliance.

At times, a user may want to extend the time between emptying their appliance, for example, when they are sleeping. Typically, a larger capacity bag is used to contain waste while the user is sleeping. In this scenario, the outlet valve is connected to the larger capacity bag by a tube, so that the bag fills with waste. The bag is typically supported on a stand while in use.

Embodiments of the present invention seek to alleviate one or more problems associated with the prior art.

According to an aspect of the invention we provide an ostomy collection volume stand for supporting a urostomy collection volume including:
a support arrangement having a first condition and a second condition, wherein in the first condition the support arrangement provides an enclosed storage volume for storing one or more urostomy collection volumes and
in the second condition the support arrangement provides a support formation which is configured to engage with a portion of a urostomy collection volume such that the support arrangement provides a support for the urostomy collection volume when it is in use.

The support arrangement may include a housing which may, in the first condition, provide the enclosed storage volume. The housing may include at least one side wall, a base for contact with a surface, and a closure. The closure may be pivotable about a connection to the wall.

The support formation may be provided by the closure and/or by a portion of the side wall which is adjacent to the closure. The support formation may include a first part which may be configured to engage a top portion of the urostomy collection volume, when in the second condition. The support formation may include a second part which is configured to engage a conduit which may be connected to the urostomy collection volume, when in the second condition. The support formation may be configured to support the top portion of the urostomy collection volume above a bottom portion of the urostomy collection volume so that, in use, waste flows towards the bottom portion, from the top portion, under gravity.

The ostomy collection volume stand may, prior to the first use in the second condition, have first and/or second parts of the support formation including a frangible connection which is breakable to provide a slit or an opening. The ostomy collection volume stand may include a tap opening. The tap opening may be located adjacent a base of the stand and/or towards a lower region of the stand.

According to a second aspect of the invention, we provide an ostomy collection volume stand for supporting a urostomy collection volume including:
a housing, and
a support formation, wherein the housing provides an enclosure in which the urostomy collection volume is locatable and the support formation is provided on or connected to the housing to support the urostomy collection volume when it is in use.

The housing substantially encloses the urostomy collection volume. The housing may include at least one side wall, a base for contact with a surface, and a closure. The housing may be generally cuboid in shape.

The support formation may be provided by the closure and/or by a portion of the side wall which may be adjacent to the closure. The support formation may include a first part. The first part may be configured to engage a top portion of the urostomy collection volume. The support formation may include a second part. The second part may be configured to engage a conduit which is connected to the urostomy collection volume

The support formation may be configured to support the top portion of the urostomy collection volume above a bottom portion of the urostomy collection volume so that, in use, waste flows towards the bottom portion, from the top portion, under gravity.

The stand may be formed of recyclable material. The stand may be formed of cardboard.

The ostomy collection volume stand may include a user graspable portion. The user graspable portion may include a frangible connection which is breakable to provide an opening.

One or more features of the ostomy collection volume stand according ot he first aspect of the invention may be combined with one or more features of the ostomy collection volume stand of the second aspect (and vice versa).

According to a third aspect of the invention we provide an ostomy collection system including:
a urostomy collection volume having an internal collection volume for storing waste and an inlet which fluidly connects to the internal collection volume, and
a stand according to either of the first or second aspects.

The urostomy collection volume may further include a conduit connected or connectable to the inlet. The urostomy collection volume nay be a flexible pouch or bag. The urostomy collection volume may include a loop or handle portion which is engageable with the support formation. As the urostomy collection volume collects waste, it may expand within the enclosed storage volume/enclosure of the stand.

### BRIEF DESCRIPTION OF THE FIGURES

In order that the present disclosure may be more readily understood, preferable embodiments thereof will now be described, by way of example only, with reference to the accompanying drawings, in which:

FIG. 1 is a perspective view of an ostomy collection volume stand, embodying the present disclosure;

FIG. 2 is a perspective view of an ostomy collection volume stand, embodying the present disclosure;

FIG. 3 is a schematic view of a ostomy collection volume;

FIG. 4 is a perspective view of an ostomy collection volume stand and urostomy collection volume, embodying the present disclosure;

FIG. 5 is a perspective view of a part of an ostomy collection volume stand and urostomy collection volume, embodying the present disclosure;

FIG. 6 is a perspective view of an ostomy collection volume stand, embodying the present disclosure;

FIG. 7 is a partial front side vide of an ostomy collection volume stand,

FIGS. 8 to 11 are various front views of embodiments of an ostomy collection volume stand.

### DETAILED DESCRIPTION OF THE DISCLOSURE

Referring to the figures, an ostomy collection volume stand 10 (known as stand 10 hereafter) for supporting a urostomy collection volume 200 (i.e. urostomy collection bag for collecting waste from a urostomy pouch) is shown. The stand 10 includes a support arrangement 100 having a first condition and a second condition. The stand 10/support arrangement 100 includes a housing 110 and a support formation 102. The housing 110 provides an enclosure 104 in which the urostomy collection volume 200 is locatable. The support formation 102 is provided on or connected to the housing 110 to support the urostomy collection volume 200 when it is in use (described in more detail below).

It should be appreciated that although this description outlines that the stand 10 is suitable for a ostomy collection volume (this is typically connected to a urostomy pouch that collects predominantly liquid waste), it is also suitable for an ileostomy collection volume (i.e. liquid waste from an ileostomy bag which typically has a higher flow rate than a urostomy). In other words, the invention relates equally to an ileostomy collection volume in addition to a urostomy collection volume (in which case, the same features of the ileostomy collection volume will interact with stand 10 described below).

In the first condition (an embodiment of which is shown in FIG. 1), the support arrangement 100 provides an enclosed storage volume 104 for storing one or more urostomy collection volumes 200. The support arrangement 100 has the housing 110 which provides the enclosure 104 in which the urostomy collection volume 200 is locatable. In other words, the housing 110 provides the enclosed storage volume when the support arrangement 100 is in its first condition.

In some embodiments (such as the example shown in FIG. 1), the housing 110 includes a side wall 112, a base 114 and a closure 116 (in this example, a pivotable lid). In the present example, the housing 110 includes four side walls 112, which enclose the space between the base 114 and the closure 116. In other words, the housing 110 is generally cuboid in shape (i.e. each adjacent surface of the housing 110 meets at substantially 90 degrees to form a regular shape). A regular shape with parallel sides may be preferred since it makes the support arrangement easier to store in its first condition. However, it should be appreciated that a housing of a different shape could be used as desired.

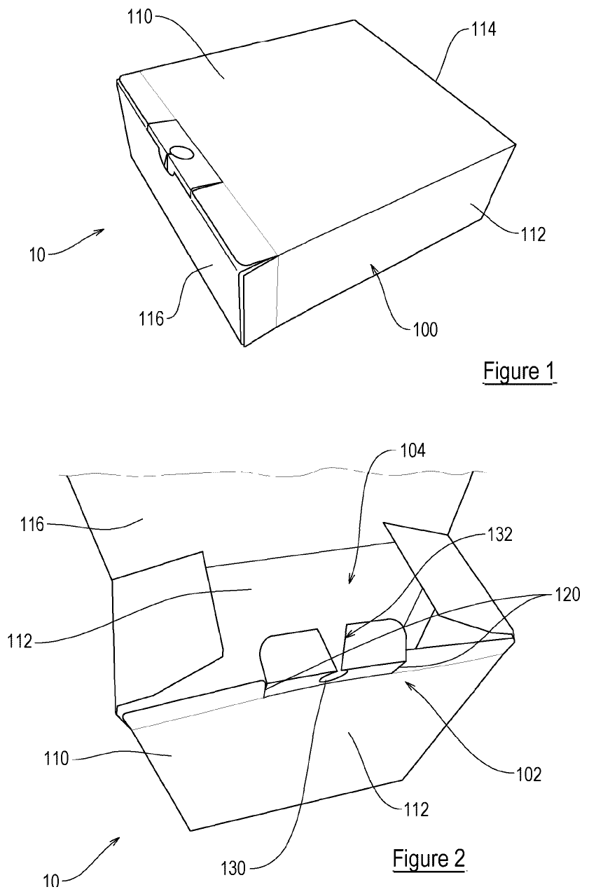

In embodiments, the closure 116 is pivotable about a connection to the side wall 112. This allows the closure 116 to be moved between a closed position (examples shown in FIGS. 1 and 6) and an open position (shown in FIGS. 2, 4 and 5). When the closure 116 is in its open position, the enclosed volume within the housing 110 is accessible. In other words, when the housing 110 is open, urostomy collection volumes 200 can be inserted/removed. When the closure 116 is in its closed position, the housing 110 forms a storage container (e.g. for use in transporting products such as urostomy collection volumes). Also when the closure 116 is in its closed position, the support formation 102 can be used to support the urostomy collection volume 200 when it is in use.

In the second condition (an embodiment of which is shown in FIG. 6), the support arrangement 100 provides a support formation 102 which is configured to engage with a portion of a urostomy collection volume 200 such that the support arrangement 100 provides a support for the urostomy collection volume 200 when it is in use.

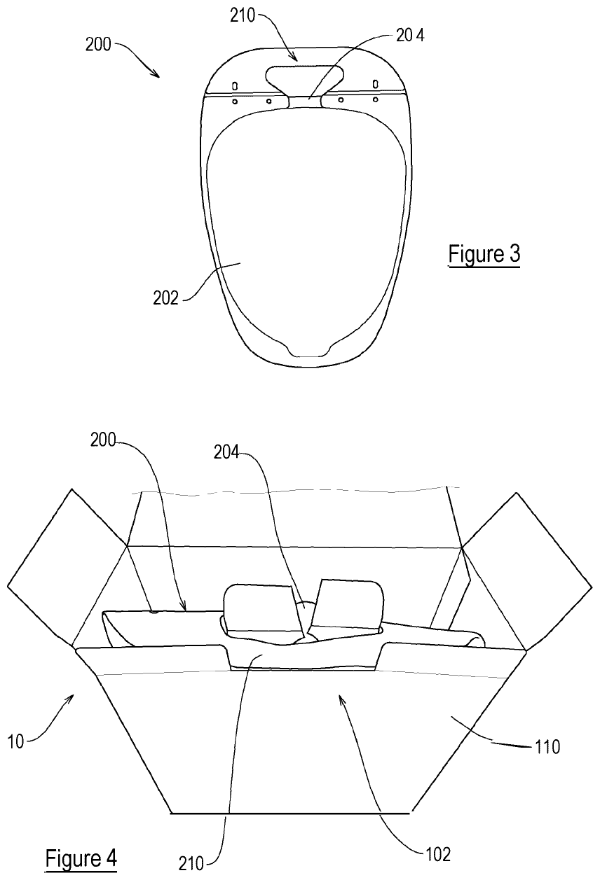

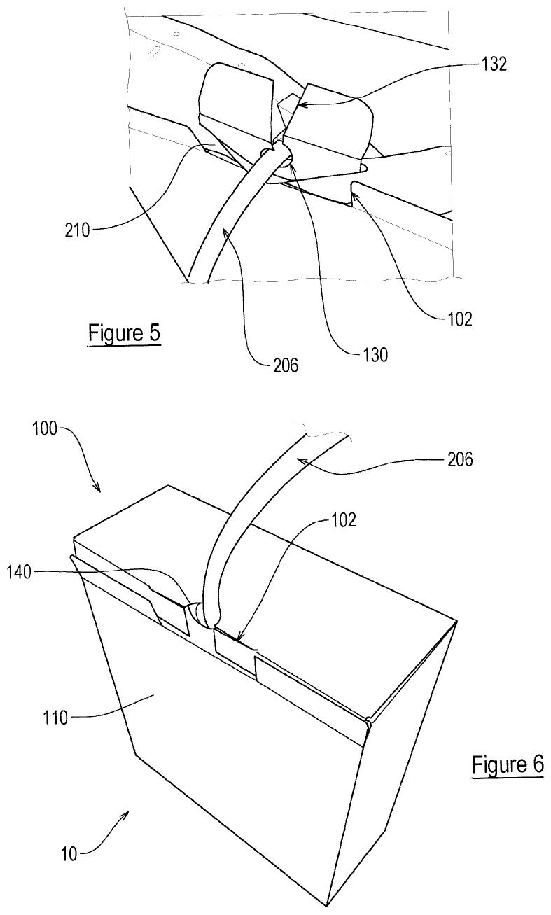

An example of a urostomy collection volume 200 is illustrated in isolation on FIG. 3. The urostomy collection volume 200 is connectable to a urostomy appliance/pouch (not shown) which is attached directly to a user (i.e. the urostomy appliance attaches about a stoma of a user). When the user wishes to extend the length of time between emptying their urostomy appliance, they attach the appliance to the urostomy collection volume 200. The urostomy collection volume 200 includes an internal volume 202 for collecting waste and an inlet 204 which fluidly connects to the internal volume 202. The urostomy collection volume 200 further includes a conduit 206, which is connected or connectable to the inlet 204 (see FIGS. 5 and 6 for an illustration of the conduit 206).

In the present example, the urostomy collection volume 200 is a flexible pouch or bag.

In some embodiments, the urostomy collection volume 200 includes a loop or handle portion 210. The loop portion 210 extends from a main body portion of the urostomy collection volume 200 and provides an opening for a user to grasp the volume 200. Furthermore, the loop portion 210 provides a part which is engageable with the support formation 102.

In some embodiments, the support formation 102 is provided by the closure 116 and/or by a portion of the side wall 112 which is adjacent to the closure 116 and/or by an interaction/cooperation between the closure 116 and the side wall 112.

In some embodiments, the support formation 102 includes a first part which is configured to engage a top portion of the urostomy collection volume 200 (i.e. the top part of the volume 200 including the loop portion 210 and inlet 204). In other words, the first part supports the loop portion 210 of the urostomy collection volume 200.

In the present example (see particularly FIGS. 2, 4 and 5), the first part includes a pair of slits 120 (or slots—i.e. a long narrow channel, opening or cut through the material). The slits 120 extend through the side wall 112 towards/away from the closure 116. Thus, an end of the slit extends to a peripheral edge of the side wall 112 (which allows the loop portion 210 to slide into each slit 120). In the illustrated example, the slits 120 are spaced apart (with a tab between) and are generally parallel. However, the slits need not be parallel to perform their function as providing a part that the loop portion 210 is supported from. For example, the slits could extend away from each other as they approach the peripheral edge of the side wall 112 and still provide a strong enough formation from which to support the loop portion 210.

In some embodiments, the support formation 102 includes a second part which is configured to engage the conduit 206 which is connected to the urostomy collection volume 200. In other words, the second part is configured to position and hold the inlet 204 and the connected conduit 206 (which leads from a connected urostomy appliance). In the present example, the second part includes an opening 130 and a channel 132. The channel 132 extends from a peripheral edge of the side wall 112 to the opening 130, so as to provide an access path to the opening 130.

In the present example, the closure 116 also includes a formation that provides auxiliary support in addition to the second part. A notch 140 is removed from a peripheral edge of the closure 116. In use, the conduit 206 extends through the notch 140 and second part to the inlet 204 of the urostomy collection volume 200. Thus, the notch 140 provides additional support to hold the urostomy collection volume 200 in position.

In some embodiments, the first and/or the second part of the support formation 102 includes a frangible connection. The frangible connection(s) is breakable prior to using the stand 10 for the first time (i.e. is involved in converting the support arrangement 100 to the second condition for the first time). In other words, a perforated portion may be provided that has to be separated before the stand 10 can be used to support the urostomy collection volume 200 for the first time.

For example, the side wall 112 where the pair of slits 120 (provided by the first part of the support formation 102) are provided is connected by a weak connection formed by perforations or the like. These perforations are separated to form the slits 120 properly and provide the formation that the loop portion 210 is supported on.

Further, in some examples, the side wall where the opening 130 and channel 132 are provided is also formed complete in the first instance. The material where the opening 130 and channel 132 are formed may be removed in order to provide the aperture through which the conduit 206 extends (and provide the channel through which the conduit 206 is moved along to reach the aperture). Again, this process is made possible by using a weakened portion such as a perforation in the side wall 112 so that the material can be removed.

In embodiments, the same is true of the notch 140 present on the closure 116. As above, this may be formed by the breaking of a frangible connection prior to the stand 10 being used to support a single urostomy collection volume 200 in use for the first time.

Thus, it should be appreciated that although the illustration of the stand arrangement 100 in FIG. 1 is shown in the configuration of the first condition, the opening 130, channel 132, pair of slits 120 and notch 140 have been formed. When the support arrangement 100 is in its first condition, intact perforations for forming the support formation 102 at a later stage will also be present.

Once the frangible connections are broken, the support formation 102 can be used to support the urostomy collection volume 200. The closure 116 is pivoted away from the side wall, so that the volume 200 can be inserted into the enclosure 104. The loop portion 210 is slid over the pair of slits 120 and the inlet 204/conduit 206 is passed through the channel 132 and into the opening 130. Once this step is complete, the closure 116 is pivoted downwards to its closed position. The notch 140 provides an opening for the conduit 206 to extend through. This forms the support arrangement's 100 second condition.

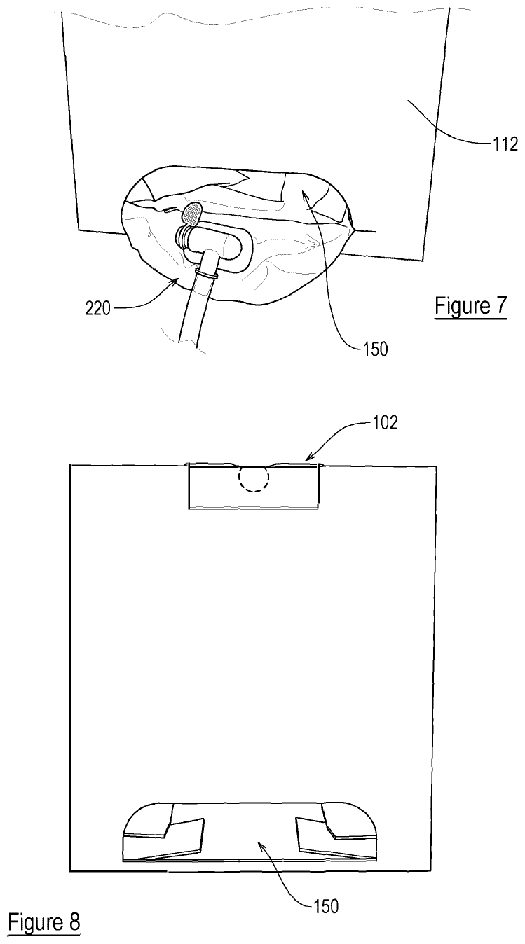

In some embodiments (see for example FIGS. 7 to 11), the stand 10 includes a tap opening 150. The tap opening 150 provides an aperture into the enclosed volume, so that a part of the collection volume 200 may extend outside from the volume and be used to empty the volume/bag supported in the stand 10. In this example, the collection volume/bag 200 has an outlet/tap/valve 220 that is openable to allow waste to exit the bag 200. As can be seen from FIG. 7 for example, the tap 220 is arranged at the bottom of the bag 200 and can be pulled through the tap opening 150 of the box so it is usable even when the bag 200 is held inside the stand 10.

The tap opening 150 is located towards the base 114. The opening 150 may be generally central along the wall (see FIGS. 7, 8 and 9 for example) or may be located in a corner (see FIGS. 10 and 11 for example).

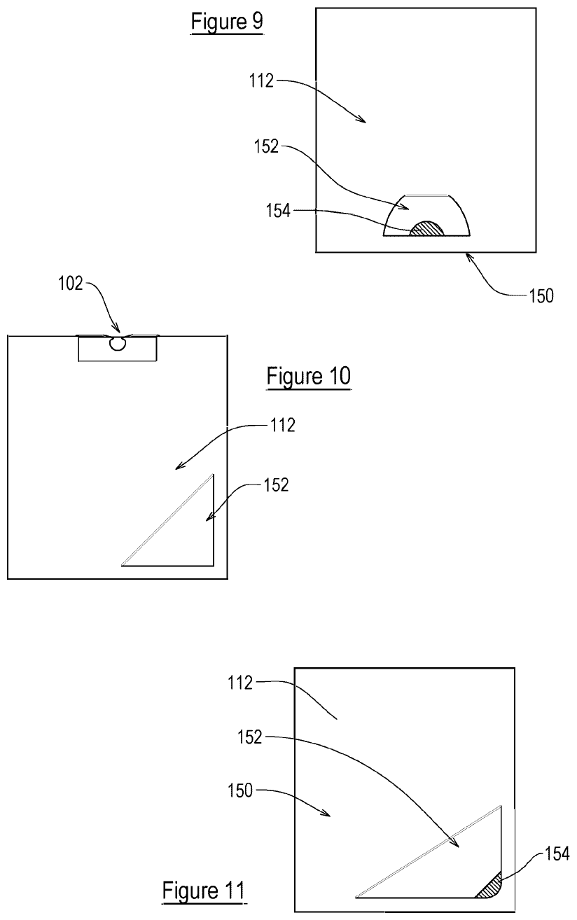

The tap opening 150 may be located on the same side of the stand 10 as the support formation 102 or it may be located on the opposite side (see, for example, FIGS. 8 and 10 versus FIGS. 9 and 11).

Further, as described above in relation to the slots/channels provided for the support formation 102, the tap opening 150 may also be provided via a frangible connection in the side wall 112. A score line or fold line may be provided in addition to the frangible connection-providing the tap opening 150 with a cover 152 over the aperture when it is not being used. The examples illustrated in FIGS. 9,10 and 11 each have a cover 152 that is connected at a top or side region to the side wall 112. Thus, the cover 152 is able to pivot down when not in use.

It should be appreciated that the cover 152 does not need to be connected at a top region. For example, the cover 152 could pivot down from a connection in a lower region and provide a surface for the bottom of the collection volume 200/the outlet 220 to rest on/be supported by.

In embodiments, the cover 152 includes a small finger grip 154, which allows a patient to grasp the cover more easily.

In some embodiments, the housing 110 includes a pair of user graspable portions (not shown). In the present example, the user graspable portions includes an opening in the side wall 112 (i.e. a hand hold is located in the wall 112). The opening may be only partially formed initially—for example, frangible connections (e.g. perforations) may be placed in the desired location at manufacture. The perforations may define an entire opening or part of an opening. In other words, the perforations may form either a complete outline of an opening to be formed or the perforations may form part of the opening, which when broken allow the material to be folded to form the opening. It should be appreciated that the user graspable portions may take an alternative configuration—e.g. an additional projection could be provided.

In use (i.e. once a single urostomy collection volume 200 is positioned as discussed above on the support formation 102), the stand 10 is placed on a surface and the conduit 206 is connected to user's urostomy appliance (i.e. the pouch or bag that attaches directly to their abdominal region around a stoma). There are multiple orientations that the stand 10 is designed to function in. Firstly (and the preferred orientation), the base 114 is placed on a generally horizontal surface (e.g. the ground) and the closure 116 forms a top surface. Thus, the stand 10 has an “upright” configuration where longest sides of the stand 10 form substantially vertical sides (and generally perpendicular to the surface upon which the stand 10 is supported on) and the base 114 and closure 116 are substantially horizontal (e.g. generally parallel to the surface the stand 10 is supported on).

In other words, the collection volume/bag 200 is hung/supported from the support formation 102 (a support located in an upper region of the stand 10) in the stand 10 and arranged to stand upright on a ground/horizontal surface, which ensures the bottom of the collection bag is positioned below the top that is connected to the support formation 102.

In embodiments, the collection volume 200 is supported in an upright (or at least inclined) position with the inlet/conduit at a top most position. The bottom of the collection volume 200 (i.e. the bottom of the bag) is supported on the base 114 of the stand 10. In other words, the stand 10 has (at least) two points of support to the collection volume 200 which are spaced vertically from each other. This allows the (in use, increasing) weight of the bag 200 to be supported by a stand that is formed of a relatively weak material (e.g. cardboard/paper based material).

Likewise, the support formation 102 and stand 10 are all formed of the same material to ensure that the stand 10 is easier to manufacture and recycle.

Alternatively, one of the side walls 112 is used as a support surface (e.g. is parallel to the surface upon which the stand 10 is supported) and the closure 116 and the base 114 is then a “side” (e.g. generally perpendicular to the surface upon which the stand 10 is supported on).

In either orientation, the support formation 102 supports the top portion of the urostomy collection volume 200 above a bottom portion of the urostomy collection volume 200. In other words, the loop portion 210 and conduit 206 will be held above the internal volume 202. This orientation in use is important so that waste flows towards the bottom portion (i.e. into the internal volume 202), from the top portion (the conduit 206 and inlet 204), under gravity. The engagement between the support formation 102 and the urostomy collection volume 200 is shown in most detail in FIG. 5 and the stand 10 as a whole with the urostomy collection volume 200 ready to collect waste is shown in FIG. 6. Further, the stand 10 is configured to bear the weight of the collection volume 200 through the loop portion 210 (the first part of the support formation 102) and bottom of the bag (on the base 114). This reduces the forces/strain through the inlet 204 part and/or conduit 206

In some embodiments, if the stand 10 is being used in its “alternative” orientation where a side wall 112 forms the bottom, an additional internal ramp may be included for the urostomy collection volume 200 to rest on. The ramp is at its highest point adjacent the support formation 102 (i.e. where the volume 200 is supported by the first and second parts) and at its lowest point towards or adjacent to the base 114.

As waste is collected in the internal volume 202, the urostomy collection volume 200 expands within the enclosed storage volume 104 of the housing 110. The centre of gravity of the combination of the stand 10 and the urostomy collection volume 200 will move over time. As waste is collected in the internal volume 202 the centre of gravity will be relatively low (the stand 10 and volume 200 are relatively light, so the waste has a proportionally large effect on the centre of gravity because it is relatively heavy). The centre of gravity will gradually move upwards as the internal volume 202 fills-however, this process will be slow as the internal volume 202 will expand outwards to fill the enclosed volume 104 and the “top” of the stand 10 remains much lighter than the bottom. This results in a stand 10 that has increased stability as it fills and reduces the risk of the stand falling over.

The stand 10 is advantageous by providing an enclosure around the urostomy collection volume 200. This protects the volume 200 from potential damage—for example, if the stand is kicked accidently then the volume 200 inside is protected from that impact.

In some embodiments, the stand 10 is formed of recyclable material—for example, a recyclable plastic, cardboard or heavy/thick paper. In some embodiments, cardboard may be preferred as a material as it is a material that balances the economics of providing a storage vessel for the urostomy collection volumes (particularly when in transport) and a strong enough material that can be used multiple times as a stand for the urostomy collection volumes when they are in use.

Thus, embodiments of the present invention provide a stand 10 that has dual functionality. In the first condition, the support arrangement 100 is used for storage and transport of products. In the second condition, the support arrangement 100 forms a stable stand 10 for use with a medical collection volume that collects waste from an ostomy pouch. Further, it is easy to dispose of in an environmentally friendly manner and can be easily recycled for further use of the material.

The stand 10 converts from a box form (i.e. a storage volume) to a stand form easily and quickly. In the first condition the box is used as storage for medical bags (i.e. one or more collection volume/bag 200 could be sent with a postal service to a patient). The user converts the stand in box form to the stand form—i.e. from the first condition to the second condition. This process includes one or more of the following steps:
breaking one or more frangible/perforated areas to form a support formation, which potentially includes forming one or more slits 120, the opening 130, the channel 132, the tap opening 150,
breaking on or more frangible or perforated portions for user graspable portions (e.g. handle openings),
locating a portion of a collection volume over/around the support formation 102 (e.g. into/through the slits 120 and around the tab formed between the slits 120),
threading the conduit 206 through the opening 130, and
positioning the stand 10 on a surface with the top (the support formation 102 and corresponding part of the collection volume 200) raised from the ground.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

### Representative Features

Representative features are set out in the following clauses, which stand alone or may be combined, in any combination, with one or more features disclosed in the text and/or drawings of the specification.

1. An ostomy collection volume stand for supporting a urostomy collection volume including:
a support arrangement having a first condition and a second condition, wherein
in the first condition the support arrangement provides an enclosed storage volume for storing one or more urostomy collection volumes and
in the second condition the support arrangement provides a support formation which is configured to engage with a portion of a urostomy collection volume such that the support arrangement provides a support for the urostomy collection volume when it is in use.
   2. An ostomy collection volume stand according to clause 1 wherein the support arrangement includes a housing which, in the first condition, provides the enclosed storage volume.
   3. An ostomy collection volume stand according to clause 2 wherein the housing includes at least one side wall, a base for contact with a surface, and a closure.
   4. An ostomy collection volume stand according to clause 3 wherein the support formation is provided by the closure and/or by a portion of the side wall which is adjacent to the closure.
   5. An ostomy collection volume stand according to clauses 3 or 4 wherein the closure is pivotable about a connection to the wall.
   6. An ostomy collection volume stand according to any one of the preceding clauses wherein, the support formation includes a first part which is configured to engage a top portion of the urostomy collection volume, when in the second condition.
   7. An ostomy collection volume stand according to clause 6 wherein the support formation includes a second part which is configured to engage a conduit which is connected to the urostomy collection volume, when in the second condition.
   8. An ostomy collection volume stand according to clauses 6 or 7 wherein the support formation is configured to support the top portion of the urostomy collection volume above a bottom portion of the urostomy collection volume so that, in use, waste flows towards the bottom portion, from the top portion, under gravity.
   9. An ostomy collection volume stand according to any one of clauses 6 to 8 wherein, prior to the first use in the second condition, the first and/or second parts of the support formation includes a frangible connection which is breakable to provide a slit or an opening.
   10. An ostomy collection volume stand for supporting a urostomy collection volume including:
a housing, and
a support formation, wherein the housing provides an enclosure in which the urostomy collection volume is locatable and the support formation is provided on or connected to the housing to support the urostomy collection volume when it is in use.
   11. An ostomy collection volume stand according to clause 10 wherein the housing substantially encloses the urostomy collection volume.
   12. An ostomy collection volume stand according to clauses 10 or 11 wherein the housing includes at least one side wall, a base for contact with a surface, and a closure.
   13. An ostomy collection volume stand according to clause 12 wherein housing is generally cuboid in shape.
   14. An ostomy collection volume stand according to clauses 12 or 13 wherein the support formation is provided by the closure and/or by a portion of the side wall which is adjacent to the closure.
   15. An ostomy collection volume stand according any one of clauses 10 to 14 wherein the support formation includes a first part which is configured to engage a top portion of the urostomy collection volume.
   16. An ostomy collection volume stand according to clause 15 wherein the support formation includes a second part which is configured to engage a conduit which is connected to the urostomy collection volume
   17. An ostomy collection volume stand according to clause 15 or 16 wherein the support formation is configured to support the top portion of the urostomy collection volume above a bottom portion of the urostomy collection volume so that, in use, waste flows towards the bottom portion, from the top portion, under gravity.
   18. An ostomy collection volume stand according to any one of the preceding clauses wherein the stand is formed of recyclable material.
   19. An ostomy collection volume stand according to any one of the preceding clauses wherein the stand is formed of cardboard.
   20. An ostomy collection volume stand according to any one of the preceding claims including a user graspable portion and, preferably, the user graspable portion includes a frangible connection which is breakable to provide an opening.
   21. An ostomy collection volume stand according to any of clauses 1 to 9 including one or more of the features in clauses 10 to 20.
   22. An ostomy collection volume stand according to any of clause 10 to 20 including one or more of the features in clauses 1 to 9.
   23. An ostomy collection system including:
a urostomy collection volume having an internal collection volume for storing waste and an inlet which fluidly connects to the internal collection volume, and
a stand according to any of clauses 1 to 22.
   24. An ostomy collection system according to clause 23 wherein the urostomy collection volume further includes a conduit connected or connectable to the inlet.
   25. An ostomy collection system according to clauses 23 or 24 wherein the urostomy collection volume is a flexible pouch or bag.
   26. An ostomy collection system according to any one of clauses 23 to 25 wherein the urostomy collection volume includes a loop or handle portion which is engageable with the support formation.
   27. An ostomy collection system according to any one of clauses 23 to 26 wherein, as the urostomy collection volume collects waste, it expands within the enclosed storage volume/enclosure.

1. An ostomy collection volume stand for supporting a urostomy collection volume including:
a support arrangement having a first condition and a second condition, wherein
in the first condition the support arrangement provides an enclosed storage volume for storing one or more urostomy collection volumes and
in the second condition the support arrangement provides a support formation which is configured to engage with a portion of a urostomy collection volume such that the support arrangement provides a support for the urostomy collection volume when it is in use.

2. An ostomy collection volume stand according to claim 1 wherein the support arrangement includes a housing which, in the first condition, provides the enclosed storage volume.

3. An ostomy collection volume stand according to claim 2 wherein the housing includes at least one side wall, a base for contact with a surface, and a closure.

4. An ostomy collection volume stand according to claim 3 wherein the support formation is provided by the closure and/or by a portion of the side wall which is adjacent to the closure, and/or wherein the closure is pivotable about a connection to the wall.

5. (canceled)

6. An ostomy collection volume stand according to claim 1 wherein, the support formation includes a first part which is configured to engage a top portion of the urostomy collection volume, when in the second condition.

7. An ostomy collection volume stand according to claim 6 wherein the support formation includes a second part which is configured to engage a conduit which is connected to the urostomy collection volume, when in the second condition, and/or wherein the support formation is configured to support the top portion of the urostomy collection volume above a bottom portion of the urostomy collection volume so that, in use, waste flows towards the bottom portion, from the top portion, under gravity, and/or wherein, prior to the first use in the second condition, the first and/or second parts of the support formation includes a frangible connection which is breakable to provide a slit or an opening.

8. (canceled)

9. (canceled)

10. An ostomy collection volume stand according to claim 1 further comprising a tap opening, and optionally the tap opening located adjacent a base of the stand and/or towards a lower region of the stand.

11. An ostomy collection volume stand for supporting a urostomy collection volume including:
a housing, and
a support formation, wherein the housing provides an enclosure in which the urostomy collection volume is locatable and the support formation is provided on or connected to the housing to support the urostomy collection volume when it is in use.

12. An ostomy collection volume stand according to claim 11 wherein the housing substantially encloses the urostomy collection volume.

13. An ostomy collection volume stand according to claim 11 wherein the housing includes at least one side wall, a base for contact with a surface, and a closure.

14. An ostomy collection volume stand according to claim 13 wherein housing is generally cuboid in shape.

15. An ostomy collection volume stand according to claim 13 wherein the support formation is provided by the closure and/or by a portion of the side wall which is adjacent to the closure, and/or wherein the base also provides a support surface for a bottom portion of the urostomy collection volume.

16. (canceled)

17. An ostomy collection volume stand according to claim 11 wherein the support formation includes a first part which is configured to engage a top portion of the urostomy collection volume.

18. An ostomy collection volume stand according to claim 17 wherein the support formation includes a second part which is configured to engage a conduit which is connected to the urostomy collection volume, and/or wherein the support formation is configured to support the top portion of the urostomy collection volume above a bottom portion of the urostomy collection volume so that, in use, waste flows towards the bottom portion, from the top portion, under gravity.

19. (canceled)

20. An ostomy collection volume stand according to claim 1 wherein the stand is formed of recyclable material, and optionally, wherein the stand is formed of cardboard.

21. (canceled)

22. An ostomy collection volume stand according to claim 1 further comprising a user graspable portion and, preferably, the user graspable portion includes a frangible connection which is breakable to provide an opening.

23. An ostomy collection system including:
a urostomy collection volume having an internal collection volume for storing waste and an inlet which fluidly connects to the internal collection volume, and
a stand according to claim 1.

24. An ostomy collection system according to claim 23 wherein the urostomy collection volume further includes a conduit connected or connectable to the inlet.

25. An ostomy collection system according to claim 23 wherein the urostomy collection volume is a flexible pouch or bag, and optionally, wherein, as the urostomy collection volume collects waste, it expands within the enclosed storage volume/enclosure.

26. An ostomy collection system according to claim 23 wherein the urostomy collection volume includes a loop or handle portion which is engageable with the support formation.

27. (canceled)
